SEQUENCE LISTING (13 sequences)

SEQ ID NO: 1 - protein, 8 aa, Mus musculus
SIINFEKL

SEQ ID NO: 2 - protein, 19 aa, Mus musculus
IINFEKLTEWTSSNVMEER

SEQ ID NO: 3 - protein, 8 aa, Mus musculus
RGYVYGQL

SEQ ID NO: 4 - protein, 19 aa, Mus musculus
SSKAQVFEHPHIQDAASQL

SEQ ID NO: 5 - DNA, 18 bp, Artificial
  Artificial antisense oligo directed against invariant chain Ii
ttggtgatccatggctct

SEQ ID NO: 6 - DNA, 18 bp, Artificial
  Second artificial oligo directed against invariant chain Ii
tggtcatccatggctcta

SEQ ID NO: 7 - DNA, 18 bp, Artificial
  Scrambled oligo having the same base composition as SEQ ID NO 5
tctcggtacctagtggtt

SEQ ID NO: 8 - DNA, 18 bp, Artificial
  Control oligonucleotide
atggatgaccaacgcgac

SEQ ID NO: 9 - DNA, 18 bp, Artificial
  Complementary oligo to SEQ ID NO 6
tagagccatggatgagga

SEQ ID NO: 10 - DNA, 42 bp, Artificial
  Oligo used in conjunction with SEQ ID NO 11 to amplify murine TRP2 mRNA
taatacgactcactatagggagacatgggccttgtgggatgg

SEQ ID NO: 11 - DNA, 84 bp, Artificial
  Oligo used in conjunction with SEQ ID NO 10 to amplify the murine TRP2 mRNA
ttttttttttttttttttttttttttttttttttttttttttttttttttttttttttttttttctaggcttcctccgtgtatc

SEQ ID NO: 12 - DNA, 44 bp, Artificial
  Oligo used in conjunction with SEQ ID NO 13 to amplify tOVA mRNA
taatacgactcactagggagacatggccatggtatacctgggtg

SEQ ID NO: 13 - DNA, 83 bp, Artificial
  Oligo used in conjunction with SEQ ID NO 12 to amplify tOVA mRNA
ttttttttttttttttttttttttttttttttttttttttttttttttttttttttttttttttaaggggaaacacatctgcc

(19) United States
(12) Patent Application Publication
(10) Pub. No.: US 2004/0161417 A1
(43) Pub. Date: Aug. 19, 2004

(54) Method of enhancing CD4+ T cell responses

(76) Inventors: Eli Gilboa, Durham, NC (US); Yangbing Zhao, Rockville, MD (US)

Correspondence Address: JENKINS & WILSON, PA, 3100 TOWER BLVD, SUITE 1400, DURHAM, NC, 27707, US

(21) Appl. No.: 10/641,316

(22) Filed: Aug. 14, 2003

Related U.S. Application Data
(60) Provisional application No. 60/402,944, filed on Aug. 14, 2002.

(51) Int. Cl. A61K 48/00; C12N 5/08

(52) U.S. Cl. 424/93.21; 435/366

(57) ABSTRACT

The present invention provides a method of enhancing CD4 T cell responses to antigens by inhibiting expression of invariant chain protein (li) in antigen presenting cells and antigen presenting cells modified by the method.

### RELATED APPLICATIONS

This application claims the benefit of U.S. Provisional Patent Application Serial No. 60/402,944, filed Aug. 14, 2002; the disclosure of which is incorporated herein by reference in its entirety.

### TECHNICAL FIELD

The present invention relates to a method of enhancing CD4⁺ T cell responses by inhibiting expression of invariant chain protein (li) in antigen presenting cells.

### BACKGROUND OF THE INVENTION

Throughout this specification, references are referred to in parenthesis. Each of these references is hereby incorporated by reference.

The dendritic cell (DC) network is a specialized system for presenting antigen to naive or quiescent T cells and consequently plays a central role in the induction of T cell, as well as B-cell, immunity (Banchereau et al., Ann. Rev. Immunol. 18:767-811 (2000)). Immunization with dendritic cells that are loaded with tumor antigens represents a powerful method of inducing antitumor immunity (Gunzer et al., Semin. Immunol. 13(5): 291-302 (2001)). An effective way of loading DC with tumor antigen is to transduce the DC with recombinant viral vectors or to transfect them with mRNA encoded tumor antigens. It has recently been shown that murine and human DC transfected with mRNA can stimulate potent CTL responses in vitro and in vivo (Boczkowski et al., J. Exp. Med. 184(2): 465-472 (1996), Nair et al., Nat. Biotechnol. 16(4): 364-369 (1998)). Treatment of tumor bearing mice with tumor RNA transfected DC led to a significant reduction in metastases and provided a survival benefit (Boczkowski et al., J. Exp. Med. 184(2): 465-472 (1996), Ashley et al., J. Exp. Med. 186(7): 1177-1182 (1997)). In a phase I clinical trial, PSA (prostate-specific antigen)-specific T cell responses were stimulated in patients with metastatic prostate cancer by immunization with PSA mRNA transfected DC. Despite the advanced nature of the disease, clinically related responses were seen at high frequency (Heiser et al., J. Clin. Invest. 109(3): 409417 (2002)). The use of mRNA transfected DC to stimulate immunity and engender protective immunity has been independently confirmed in murine (Koido et al., J. Immunol. 165(10): 5713-5719 (2000), Zhang et al., Hum. Gene Ther. 10(7): 1151-1161 (1999), Granstein et al., J. Invest. Dermatol. 114(4): 632-636 (2000)) and human (Heiser et al., Cancer Res. 61(8): 3388-3393 (2001), Saeboe-Larssen et al., J. Immunol. Methods 259(1-2): 191-203 (2002), Strobel et al., Gene Ther. 7(23):2028-2035 (2000), Su et al., Eur. J. Immunol. 31(3):947-958 (2001), Van Tendeloo et al., Blood 98(1):49-56 (2001), Weissman et al., J. Immunol. 165(8):4710-4717 (2000)) studies. There is accumulating evidence, when functional criteria are applied, that mRNA transfection represents a superior method for loading DC with antigens (Saeboe-Larssen et al., J. Immunol. Methods 259(1-2):191-203 (2002), Strobel et al., Gene Ther. 7(23):2028-2035 (2000), Van Tendeloo et al., Blood 98(1):49-56 (2001), Weissman et al., J. Immunol. 165(8):4710-4717 (2000)). A key advantage of loading DC with mRNA is that mRNA can be amplified from a few cells and hence a sufficient, possibly unlimited, amount of antigen can be generated from a small amount of tumor tissue (Heiser et al., J. Immunol. 166(5):2953-2960 (2001), Boczkowski et al., Cancer Res. 60(4):1028-1034 (2000)). Thus, vaccination with tumor-derived mRNA transfected DC offers an effective and broadly applicable modality for the treatment of disseminated metastatic cancer which does not require the characterization of the relevant antigenic profile from each patient and will not be limited by tumor tissue availability for antigen preparation.

CD8⁺ cytotoxic T cells are an important effector arm in the antitumor immune response and the induction of potent CTL responses has been a major goal in developing immunotherapeutic strategies for cancer (Melief et al., Adv. Immunol. 75:235-282 (2000), Rosenberg, Immunity 10(3):281-287 (1999)). Yet, accumulating evidence strongly suggests that the CD4 T cell responses also play a critical role in tumor immunity (Wang, Trends Immunol. 22(5):269-276 (2001), Pardoll and Topalian, Curr. Opin. Immunol. 10(5):588594 (1998), Toes et al., J. Exp. Med. 189(5):753-756. (1999)). CD4 T cells provide important functions for the induction, persistence, and expansion of CD8 CTL (Kalams and Walker, J. Exp. Med. 188(12):2199-2204 (1998), Zajac et al., Curr. Opin. Immunol. 10(4):444-449 (1998), Frasca et al., Crit. Rev. Immunol. 18(6):569-594 (1998)). In addition, CD4 T cells, via secretion of effector cytokines such as IFN-γ, sensitize tumor cells to CTL (cytotoxic T lymphocyte) lysis via upregulation of MHC class I molecules and other components of the endogenous presentation pathway. CD4 cells stimulate the innate arm of the immune system at the tumor site and inhibit local angiogenesis (Qin and Blankenstein, Immunity 12(6):677-686 (2000), Mumberg et al., Proc. Natl. Acad. Sci. USA 96(15):8633-8638 (1999)). The importance of the CD4 T cell response in tumor immunity was highlighted in murine studies showing that CD4 T cells can eradicate tumor in the absence of CD8 T cells (Mumberg et al., Proc. Natl. Acad. Sci. USA 96(15):8633-8638 (1999), Wan et al., Cancer Res. 60(12.):3247-3253 (2000), Levitsky et al., J. Exp. Med. 179(4):1215-1224 (1994), Hock et al., J. Exp. Med. 174(6):1291-1298 (1991), James et al., Immunology 72(2):213-218 (1991), Greenberg et al., J. Exp. Med. 161(5):1122-1134 (1985)). CD4 cells may constitute the dominant effector arm (compared to CD8 T cells) in the antitumor response (Hung et al., J. Exp. Med. 188(12):2357-2368 (1998)). An optimal antitumor immune response may, therefore, require the concomitant activation of both the CD4 and CD8 T cell arms of the immune response.

Endogenously expressed antigens, such as antigens expressed in DC transfected with mRNA, will be channeled preferentially into the class I processing pathway to activate the CD8 T cell arm of the immune response (Yewdell et al., Adv. Immunol. 73:1-77 (1999)). Notwithstanding the fact that subsets of antigens synthesized in the cytoplasm which are transported to endocytic/lysosomal compartments can generate peptides for loading of class II molecules and stimulate, albeit weak, CD4 T cell responses (Lechler et al., Immunol. Rev. 151:51-79 (1996)), vaccination with mRNA transfected DC does not normally generate potent CD4 T cell responses. Wu et al. have shown that it is possible to redirect endogenously expressed antigens into the class II presentation pathway by appending a leader sequence to the amino end and a lysosomal sorting signal derived from the human lysosomal-associated membrane protein (LAMP-1) to the carboxyl end of the endogenously expressed antigen (Wu et al., Proc. Natl. Acad. Sci. USA 92(25):11671-11675 (1995); Lin et al., Cancer Res. 56(1):21-26 (1996)). This approach has been shown to enhance the in vitro generation of CD4 T cell responses against CEA (carcinoembryonic antigen) and TERT (Nair et al., Nat. Biotechnol. 16(4):364-369 (1998)) (Shu et al., Cancer Res. in press 62 (2002)). Engineering lysosomal/endosomal targeting signals is, however, not applicable to immunization with an antigenic mixture such as tumor derived mRNA.

MHC class I negative tumor cells transfected with class II cDNA expression plasmids exhibit enhanced antitumor immunogenicity in mice, presumably due to the acquired ability to present class II restricted antigens and stimulation of tumor specific CD4 T cell responses (James et al., Immunology 72(2):213-218 (1991), Ostrand-Rosenberg et al., J. Immunol. 144(10):4068-4071 (1990), Chen and Ananthaswamy, J. Immunol. 151(1):244-255 (1993), Armstrong et al., Proc. Natl. Acad. Sci. USA 94(13):6886-6891 (1997)). Interestingly, co-expression of the Invariant chain (li) (Clements et al., J. Immunol. 149(7):2391-2396 (1992)) or the class II transactivator (CIITA) or treatment of the tumor cells with IFN-γ abrogates the immunogenicity of the class II transfected tumor cells (Armstrong et al., Proc. Natl. Acad. Sci. USA 94(13):6886-6891 (1997)). This is likely because expression of CIITA or incubation with IFN-γ upregulates both class II as well as li in many tumor cells. This observation was interpreted to reflect the natural role of li to prevent the association of endogenously derived class II peptides in the endosome or Golgi compartments with the nascent class II molecules (Bertolino and Rabourdin-Combe, Crit. Rev. Immunol. 16(4):359-379 (1996)). This is consistent with the finding that presentation of endogenous peptides is favored in cells expressing class II molecules in the absence of li (Armstrong et al., Proc. Natl. Acad. Sci. USA 94(13):6886-6891 (1997), Dodi et al., Eur. J. Immunol. 24(7):1632-1639 (1994), Long et al., J. Immunol. 153(4):1487-1494 (1994), Bodmer et al., Science 263(5151):1284-1286 (1994)).

Transfection of tumor cells with patient-specific class II alleles is not a practical approach to stimulate antitumor CD4 T cell immunity in cancer patients. Recently, Humphreys and colleagues have proposed a more general strategy whereby class II as well as li expression is induced in tumor cells by either transfection with a CIITA expression plasmid or treatment with IFN-γ followed by selective downregulation of li expression using antisense oligonucleotides directed against li (li AS ODNs) (Xu et al., Trends Biotechnol. 18(40):167-172 (2000)). Qiu et al have shown that a class II negative tumor (Sal) transfected with CIITA or treated with IFN-γ and then incubated with li AS (antisense) ODNs (oligonucleotides) exhibited enhanced antitumor immunity against a challenge with wild type tumor (Qiu et al., Cancer Immunol. Immunother. 48(9):499-506 (1999)). This approach is, however, limited by the availability of tumor tissue and the difficulty of transfecting primary human tumors. In addition, different tumors vary in their responsiveness to IFN-γ mediated induction of class II expression.

U.S. Pat. No. 5,726,020 discloses reverse gene constructs that hybridize to invariant chain mRNA and inhibit its translation. The constructs are useful in the generation of antigen presenting cells, especially malignant antigen presenting cells, such as leukemia, lymphoma, and melanoma which can present autodeterminant peptides in association with MHC Class II molecules. U.S. Pat. No. 6,368,855 also discloses MHC class II antigen presenting cells containing oligonucleotides which inhibit li protein expression.

While these methods are useful to enhance MHC Class II presentation of endogenous antigens, they are limited to the naturally occurring antigens of the antigen presenting cells. Thus, there remains a need for new methods to enhance the generation of CD4 T cell responses to a wide variety of antigenic determinants from tumors and infectious agents.

### SUMMARY OF THE INVENTION

The present invention relates to a method of enhancing the generation of CD4 T cell responses to antigens presented by antigen presenting cells (APC), such as dendritic cells (DC) and including APC transfected with tumor-derived mRNA. The invention demonstrates that inhibition of li expression in mRNA transfected APC leads to enhanced presentation of class II-restricted mRNA-encoded epitopes, increased stimulation of CD4 T cell response and potentiation of antitumor immunity. Since vaccination with tumor mRNA transfected APC does not require the identification of specific tumor antigens for each patient and is not limited by tumor tissue availability, the present invention provides a broadly useful approach to augment antitumor CD4 T cell immunity, together with CD8 T cell immunity, in cancer patients. The present method is also applicable to the induction of immunity against other endogenous antigens, such as those of intracellular pathogens such as viruses and bacteria.

In one aspect of the invention, there is provided an immunogenic composition comprising antigen presenting cells transfected with mRNA encoding at least one antigen. The antigen presenting cells further comprise an inhibitor of invariant chain (li). The antigen can also be introduced into the cell using a viral vector.

The inhibitor of invariant chain can be an antisense oligonucleotide, an RNAi molecule, a ribozyme that specifically cleaves the li mRNA, or a molecule which inhibits the interaction of li protein with MHC class II molecules.

The transfected mRNA may encode at least one tumor antigen or at least one pathogen antigen. Alternatively total tumor or pathogen RNA can be used.

In another aspect of the invention, an antigen presenting cell transfected with mRNA encoding at least one antigen and expressing on its surface the antigen in association with an MHC Class II molecule is provided. In these cells invariant chain expression or function is inhibited.

The present invention also provides a method of enhancing MHC class II presentation of epitopes encoded by mRNA transfected into antigen presenting cells. The method comprises treating the transfected antigen presenting cells with an inhibitor of invariant chain.

In another aspect, a method for enhancing a CD4 T cell response to an antigen is provided which comprises transfecting antigen presenting cells with mRNA encoding the antigen and an inhibitor of li expression.

Additional objects and advantages of the present invention will be clear from the description that follows.

### BRIEF DESCRIPTION OF THE DRAWINGS

Preferred embodiments of the invention are described below with reference to the drawings in which:

FIG. 1A illustrates the flow cytometric analysis of cells transfected with Green Fluorescent Protein (GFP);

FIG. 1B demonstrates presentation of the dominant MHC class I-restricted OVA epitope in DC electroporated with OVA (ovalbumin) mRNA or GFP mRNA;

FIG. 2A demonstrates the inhibition of invariant chain expression (CD74) in DC incubated with antisense oligonucleotides;

FIG. 2B illustrates two color staining of DC with PE-labeled anti-CDIIc and FITC-labeled anti-CD74, CD40, CD80, CD86 or MHC class II (I-Ab) antibodies;

FIG. 3A illustrates presentation of the dominant MHC class II OVA epitopes;

FIG. 3B illustrates presentation of dominant class I OVA epitopes using corresponding T-hybridomas;

FIG. 4A illustrates the induction of CD4⁺ T cell responses in mice immunized with OVA mRNA transfected DC;

FIG. 4B illustrates the induction of cytotoxic T cell (CTL) responses in mice immunized with OVA mRNA transfected DC;

FIG. 5A illustrates enhancement of protective antitumor immunity in mice immunized with li AS ODN treated DC;

FIG. 5B illustrates the effect on tumor volume of immunization with DC transfected with either TRP2 mRNA, Flu M1 mRNA or total B16/F10.9 tumor RNA (F10.9 RNA) and treated with li AS (AE40) or control (SE40) ODNs; and

FIG. 6 illustrates enhancement of tumor regression in mice immunized with li AS ODN-treated DC.

### DETAILED DESCRIPTION OF THE INVENTION

Antigen-loaded antigen presenting cells (APC) can be used to stimulate protective immunity against infectious agents and tumors. Antigens expressed in APC transfected with a nucleic acid(s) encoding the desired antigen(s) are channeled preferentially into the class I processing pathway to generate potent CD8 cytotoxic T lymphocyte (CTL) responses. Generation of CD4 T cell responses is, however, limited. The present invention provides a method of enhancing the stimulation of CD4 T cell responses by antigen-loaded APC by inhibiting expression of invariant chain (li).

APC suitable for use in the present invention are, advantageously, professional APC, such as dendritic cells (DC), macrophage/monocytes, and B cells. However, any APC can be used (e.g., endothelial cells or artificially generated APCs). While it is preferred that the cells administered to the patient be derived from that patient (autologous), APC can be obtained from a matched donor or from a culture of cells grown in vitro. Methods for matching haplotypes are known in the art.

Methods of introducing tumor or pathogen antigen are known in the art and the invention includes any method in which the antigen (or portion thereof) reaches the cytoplasm of the APC so that it would normally be processed by the endogenous pathway and preferentially stimulate CD8 CTL responses. For example, cells can be transfected with mRNA encoding antigen. Alternatively, viral vectors can be used to introduce antigen into the cells. Infection with a pathogen will also introduce pathogen antigens into the cell. Other methods of introducing antigens into cells as described in the art can also be used.

Methods of loading APC with tumor or pathogen nucleic acid encoding the desired antigen are described, for example, in U.S. Pat. Nos. 5,853,719 and 6,306,388, the contents of which are incorporated herein by reference. Such methods include conventional transfection methods (lipid-mediated transfection, electroporation and calcium phosphate transfection). Also described are methods of isolating and amplifying, or otherwise producing (e.g., chemically or recombinantly), antigen encoding nucleic acid. The nucleic acid (e.g., RNA) can be provided to the APC, for example, as purified nucleic acid or as a fractionated preparation. Tumor-specific or pathogen-specific nucleic acid (as defined in U.S. Pat. Nos. 5,853,719 and 6,306,388) can be used.

The present invention provides methods and constructs which enhance the induction of CD4 T cell responses to antigens that would normally preferentially stimulate a CD8 T cell response. This is achieved by treating antigen presenting cells with an inhibitor of the invariant chain (li inhibitor). The term, li inhibitor is used herein to refer to any agent which affects the quantity or activity of li in antigen presenting cells. An li inhibitor may act by downregulating expression of invariant chain or by preventing interaction of li with MHC Class II molecules.

Inhibition of li expression in the antigen-loaded APC of the invention can be effected, for example, by employing antisense technology using specific oligonucleotides (e.g., chemically synthesized) or reverse gene constructs (e.g., recombinant expression vectors in which a nucleic acid encoding a sequence of the li gene in an orientation such that mRNA is produced that is antisense to, for example, a coding or regulatory region of the li gene). See, for example, U.S. Pat. Nos. 5,726,020 and 6,368,855, and U.S. Patent Application 2002 0086421 A1, the contents of each being incorporated herein by reference. As indicated in U.S. Pat. No. 6,368,855, the li RNA sequences that can be targeted in accordance with these approaches include protein coding segments (exons), intervening segments (introns), splice sites, the initiation site or sites 5′ thereto (e.g., CAP sites or other regulatory sites), or sites 3′ to the coding sequence (e.g., 3′ polyA-specific regulatory sites).

The antisense oligonucleotides or constructs can be introduced into APC by incubating the cells with oligonucleotides/constructs under conditions such that spontaneous uptake occurs. Alternatively, uptake can be assisted by the use of lipids/liposomes, electroporation, or other techniques.

In addition to the antisense oligonucleotides and antisense constructs described above, other li inhibitors are also useful in the present invention. Examples of such alternative inhibitors are described, for example, in U.S. Pat. No. 6,368,855 and include copolymers of nucleotide bases that hybridize to the li gene and organic molecules that alter the interaction of li protein with the MHC class II molecule so as to enhance binding of endogenous antigenic determinants to MHC class II molecules. Furthermore, proteases or nucleic acids encoding them can be used to inhibit the activity of invariant chain molecules.

RNA interference can also be used to effect inhibition of li expression (see, for example, Lagos-Quintana et al., Science 294:853-858 (2001); Lau et al., Science 294:858-862 (2001); Lee and Ambros, Science 294:862-864 (2001); Sharp, Genes Dev. 15:485-490 (2001); Elbashir et al., Nature 411:494-498 (2001); Fire et al., Nature 391:806-811 (1998); Hammond et al., Nature 404:293-295 (2000)). RNAi (RNA interference) refers to the introduction of homologous double stranded RNA (dsRNA) to specifically target a gene's product, resulting in null or hypomorphic phenotypes. RNA interference is a general mechanism for silencing the transcript of an active gene, mRNA. This process of post-transcriptional gene silencing is initiated by small interfering RNA (siRNA), a double-stranded form of RNA that contains 21 to 23 bp and is highly specific. It is remarkably potent and only a few dsRNA molecules per cell are required for effective interference. RNAi can be used to specifically target and silence the invariant chain transcript.

In accordance with the invention, APC can be antigen-loaded first or the li inhibitor can be introduced first. Alternatively, the introductions can be simultaneous.

The amount of li inhibitor introduced into the APC can depend, for example, on the nature of the inhibitor and the APC. One skilled in the art can readily determine optimum amounts.

The APC of the present invention are useful in the treatment of an existing tumor or in the prevention of tumor formation in a patient (a human or non-human animal) (e.g., melanoma tumors, bladder tumors, breast cancer tumors, colon cancer tumors, prostate cancer tumors, and ovarian cancer tumors). The APCs can be provided in an immunogenic composition comprising APCs carrying a nucleic acid encoding an antigen and an li inhibitor. The immunogenic composition preferably includes an acceptable carrier or excipient. Generally, it is advantageous that treatment begins when the tumor burden is low, and continues until the cancer is ameliorated. However, the invention is suitable for use even after a tumor has formed. In treating a patient in accordance to the invention, the optimal dosage depends on factors such as the weight of the patient, the severity of the cancer, and the nature of the antigen targeted. When APC are used, a dosage of 10⁵ to 10⁸ cells/kg body weight, preferably 10⁶ to 10⁷ cells/kg body weight is administered in a pharmaceutically acceptable excipient to the patient. The cells can be administered, for example, using infusion techniques that are commonly used in cancer therapy. One skilled in the art can readily determine the optimal dosage and treatment regime for a particular patient by monitoring the patient for signs of disease and adjusting the treatment accordingly. The treatment can also include administration of mitogens (e.g., phytohemagglutinin) or lymphokines (e.g., IL-2 or IL-4) to enhance T cell proliferation. The method of the invention can also be used in combination with other modalities for treating cancer, such as radiation, chemotherapy and anti-angiogenic therapy.

In an exemplary embodiment of the invention, antigen presenting cells are transfected with mRNA encoding at least one tumor antigen. The APC may be transfected with total tumor mRNA. The function or expression of the invariant chain is suppressed to enhance the presentation of tumor antigen epitopes in association with MHC Class II molecules. In one embodiment, translation of the li mRNA is blocked by antisense RNA or RNAi.

In accordance with the invention, APC can also be loaded so as to be useful in the treatment of infection by pathogens, such as Salmonella, Shigella, Enterobacter, human immunodeficiency virus, Herpes virus, influenza virus, poliomyelitis virus, measles virus, mumps virus, or rubella virus. As above, one skilled in the art can readily define optimum dosing strategies.

The modified antigen presenting cells of the present invention may also be useful in the treatment of certain autoimmune diseases. Bischof et al. have shown that genetically modified li proteins, in which the CLIP core region has been replaced by a T helper epitope of myelin basic protein, can be processed and presented in vitro to T cell clones by HLA-DR-expressing cell lines. When delivered in a tolerogenic manner by i.p. injections, the modified protein eliminated the proliferative capacity of antigen-specific T cells more potently than equivalent amounts of the peptide. They also found that similar in vivo tolerance induction by the protein efficiently prevented the subsequent evolvement of autoimmunity (Bischof et al., Proc. Natl. Acad. Sci. USA. 98(21):12168-12173 (2001). Tolerance to self antigens has been shown by others to be maintained by a CD4 T cell subset known as regulatory CD25⁺ CD4⁺ T cells. Thus, DC transfected with mRNA encoding self antigens in which the li chain activity has been altered may prove useful in the generation of regulatory T cells.

It is clearly apparent that the immunogenic compositions and methods of the present invention are useful in the modulation of immune responses in both humans and animals and in the treatment of diseases in both humans and animals. The term “immunogenic” is used herein to mean a shift in the immune response. There may be an enhancement or downregulation of the numbers or activities of certain immune cells or there may be a switch in the types of cells involved.

The present invention also provides kits for the preparation of antigen presenting cells. The kit comprises a supply of antigen-encoding mRNA and li inhibitor. The kit may also include agents which facilitate the uptake of the antigen-encoding mRNA and inhibitor. Directions for use are also optionally included. In a preferred embodiment, the mRNA is tumor mRNA.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances may suggest or render expedient. Although specific terms have been employed herein, such terms are intended in a descriptive sense and not for the purposes of limitation. Methods of immunology and molecular biology referred to but not explicitly described in this disclosure and examples are reported in the scientific literature and are well known to those skilled in the art.

### EXAMPLES

### Example 1

### MHC Class I Presentation of Antigen

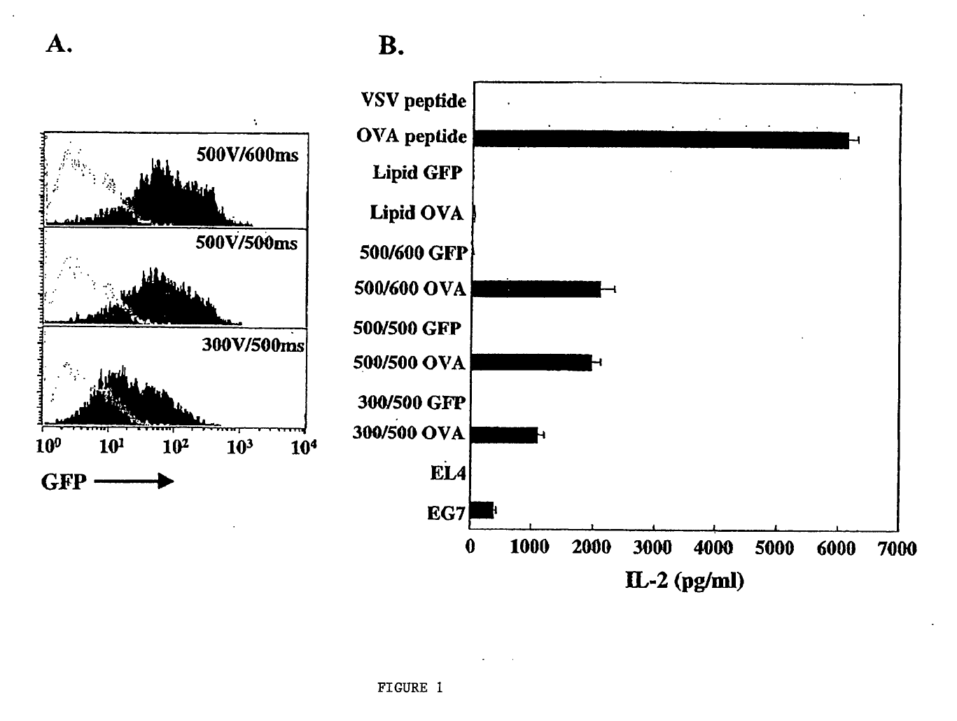

DC were electroporated with GFP (green fluorescent protein) mRNA using varying conditions of voltage and time (ms-milliseconds) and 24 hours later expression of GFP was determined by flow cytometry. mRNA electroporation is a highly efficient method to express and present antigens by human DC (Saeboe-Larssen et al., J. Immunol. Methods 259(1-2):191-203 (2002), Van Tendeloo et al., Blood 98(1):49-56 (2001)). As shown in FIG. 1A, bone marrow derived murine DC are efficiently transfected by GFP mRNA. Efficiency of transfection is influenced by the voltage and duration of electroporation. FIG. 1B demonstrates presentation of the dominant MHC class I-restricted OVA epitope in DC electroporated with OVA (ovalbumin) mRNA or GFP mRNA. Presentation was measured using the RF33.70 T-hybridoma. OVA presentation in DC transfected in the presence of the lipid DMRIE-C (Boczkowski et al., J. Exp. Med. 184(2):465-472 (1996)) or pulsed with the MHC class I restricted OVA or VSV peptides was also determined. FIG. 1B shows that DC electroporated with mRNA encoding the chicken ovalbumin (OVA) cDNA process and present the dominant OVA epitope to a corresponding T-hybridoma. Efficiency of OVA presentation by the transfected DC (FIG. 1B) appears to correlate with the efficiency of electroporation as measured by gene expression (FIG. 1A).

### Example 2

### Inhibition of Invariant Chain Expression in DC Treated with Antisense Oligonucleotides.

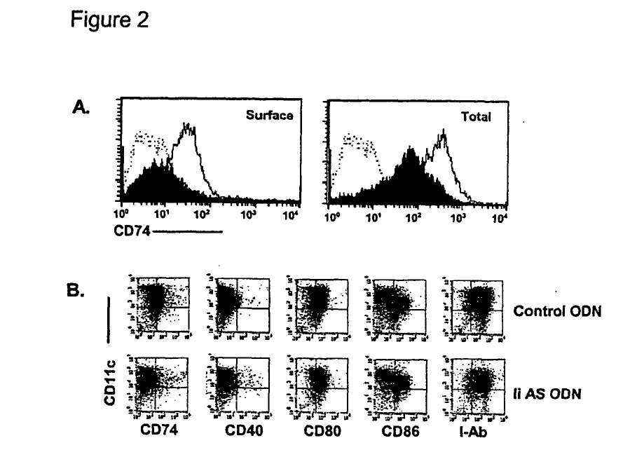

Two phosphorothioate modified antisense oligonucleotides (ODNs) described by Qiu et al (Qiu et al., Cancer Immunol. Immunother. 48(9):499-506 (1999)) were used to inhibit li expression in murine DC. Day 7 DC were electroporated with OVA mRNA and 50 nM of li AS ODN (AE40) or control ODN (SE40). To measure cell surface li expression, 48 hours later, cells were stained with FITC-labeled anti-mouse CD74 (li) antibody. The results are shown in FIG. 2A. The filled histogram shows the results obtained with the li ASO DN and the open histogram illustrates the results obtained with the control ODN. The dotted line histogram represents DC stained with FITC-labeled isotype control antibody. To measure total li expression, cells were permeabilized before antibody staining. FIG. 2A shows that surface expression of li (CD74) is significantly reduced in DC incubated with an antisense, but not control, ODN, whereas a partial inhibition was seen when both intracellular and cell surface expression of li were measured. Similar effects were seen with a second antisense and control ODN. The difference between the extent of inhibition of cell surface and intracellular li expression suggests that the intracellular pool of li has a slower turn over rate compared to the cell surface pool.

FIG. 2B illustrates two-color staining of DC with PE-labeled anti-CDIIc and FITC-labeled anti-CD74, CD40, CD80, CD86, or MHC class II (I-Ab) antibody. FIG. 2B demonstrates the specificity of the li antisense ODNs. No inhibition of CD40, CD80, CD86 or MHC class II I-A was seen in DC incubated with li specific antisense ODNs.

### Example 3

### Presentation of MHC Class I and Class II OVA Epitopes

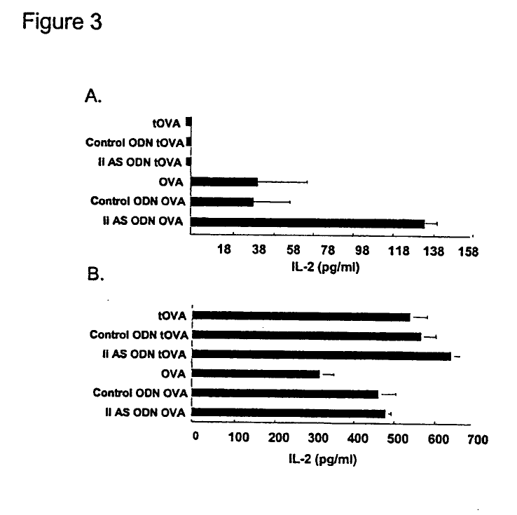

Inhibition of li synthesis enhances MHC class II presentation of OVA by DC transfected with OVA mRNA. DC were transfected with OVA mRNA or a truncated OVA mRNA (tOVA) from which sequences corresponding to the first 40 amino acids of the OVA protein were deleted. Where indicated, the OVA mRNA transfected DC were also incubated with li AS (AE40) or control (SE40) ODNs. Processing and presentation of the class I and class II epitopes by the OVA mRNA-transfected DC was determined using T-hybridomas specific to each epitope. C57BL/6 (H-2b) mice present an H-2 Kb class I restricted dominant epitope (SIINFEKL; SEQ ID NO: 1) and an I-Ab restricted class II dominant epitope (IINFEKLETEWTSSNVMEER; SEQ ID NO: 2). As shown in FIG. 3A, no presentation of the class II epitope was seen from a truncated form of OVA (tOVA) from which the first 40 amino acids containing the leader sequence were removed. This is consistent with the observations that class II presentation of endogenous antigens is confined primarily to antigens which can access the endocytic compartments (Lechler et al., Immunol. Rev. 151:51-79 (1996)). In contrast, for the native and secreted form of OVA (OVA) the class II epitope is processed for presentation to the class II restricted T-hybridoma. Incubation of the OVA mRNA transfected DC with an li antisense, but not control, ODN enhanced the presentation of the class II OVA epitope. The antisense mediated enhancement of class II presentation was reproducibly seen in multiple experiments with this and a second pair of control and antisense ODNs. As shown in FIG. 3B, presentation of the dominant OVA class I epitope was not significantly effected by the antisense or control ODNs. The illustrative experiment in FIG. 3 shows that transient inhibition of li expression in cultured DC enhances the presentation of class II, but not class I, epitopes from the endogenously expressed OVA antigen.

### Example 4

### Induction of CD4⁺ T Cell Responses and Cytotoxic T Cell (CTL) Responses in Mice Immunized with OVA mRNA Transfected DC in Which li Expression was Inhibited.

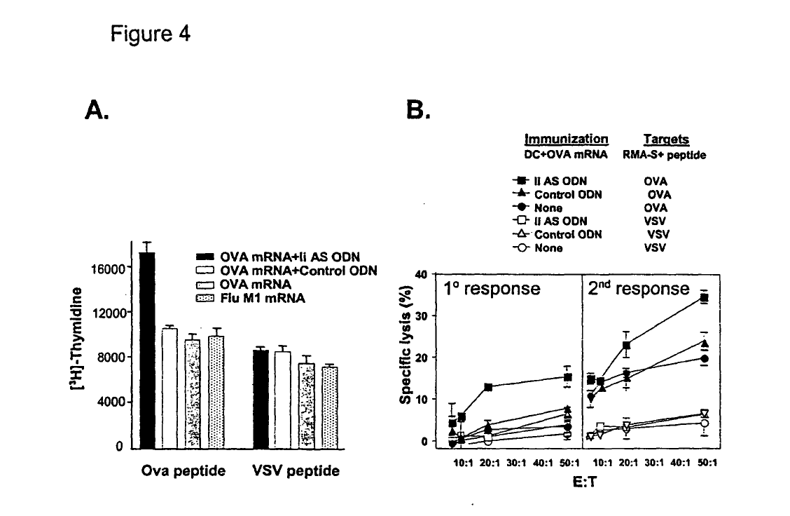

To determine whether inhibition of li expression can influence the stimulation of OVA-specific CD4 T cell responses in vivo, mice were immunized with OVA mRNA transfected DC treated with control or li antisense ODNs and the induction of OVA specific CD4 T cells was measured in the splenocytic population of the immunized mice using a standard proliferation assay. FIG. 4A illustrates the results of a CD4⁺ T cell proliferation assay. Mice were immunized i.v. with 2-4×10⁵ DCs transfected with either OVA mRNA or influenza matrix (Flu M1) mRNA in 200 μl of PBS. Where indicated, the OVA mRNA transfected DC were also incubated with li AS (AE40) or control (SE40) ODNs. Splenocytes were harvested after 8 days and CD4⁺ T cells were isolated using StemSep Murine CD4 Negative Isolation Column (Stemcell Technologies Inc.). CD4′ T cells were co-cultured with I-Ab-restricted OVA258-276 or VSV peptides pulsed DC for 3 days. 3H-thymidine incorporation was measured for 17 hours prior to harvest. FIG. 4A shows that only mice immunized with li AS ODN treated, OVA mRNA transfected DC exhibit an OVA specific CD4 T cell proliferative response. OVA mRNA transfected DC alone or treated with a control ODN did not stimulate a detectable level of CD4 T cell response above background. The specificity of the response is demonstrated by the fact there was no proliferation above background when mice were immunized with influenza matrix (Flu M1) mRNA transfected DC or stimulated in vitro with a control (VSV) peptide. The high basal level of CD4 T cell proliferation seen in this experiment may represent an anti-FCS response stemming from the use of DC cultured in the presence of FCS (Inaba et al., J. Exp. Med. 176(6):1693-1702 (1992)).

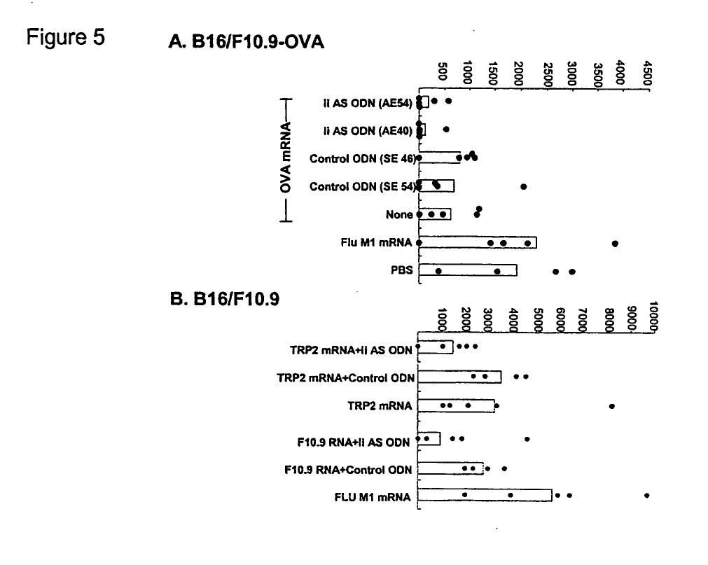

To determine whether the modest induction of a CD4 T cell response seen in mice immunized with the li antisense treated DC is of physiological relevance, the effect of li inhibition on priming of CTL response and stimulation of tumor immunity was evaluated. The role of CD4 T help in CTL induction is well documented. Therefore, experiments were undertaken to determine whether incubation of OVA mRNA transfected DC with li antisense ODNs can influence the magnitude of the OVA CTL primed in the immunized mice. Mice were immunized with 2.5×10⁵ OVA mRNA transfected DCs incubated with li AS (AE40) or control (SE) ODNs, as indicated. Splenocytes isolated 8 days post immunization were either tested directly for OVA CTL (1° response) or first incubated in vitro in the presence of OVA mRNA transfected DC and then tested for OVA CTL (2nd response). RMA-S cells pulsed with the MHC class I restricted OVA or VSV peptides were used as targets. As shown in FIG. 4B, an increase in OVA CTL was seen if the DC were treated with the li AS ODNs but not with control ODNs or not treated with ODNs. This li antisense dependent increase in CTL induction was reproducibly observed using the li antisense ODN shown in FIG. 5 and a second li antisense ODN. Detection of an OVA specific CTL in mice immunized with OVA mRNA transfected DC requires in vitro stimulation of splenocytes to expand the low frequency of OVA CTL induced in vivo to a level that can be detected by the standard cytotoxicity assay used in this experiment. Interestingly, OVA specific CTL could be detected directly without ex vivo stimulation when the immunizing DC were treated with li antisense, but not control, ODNs, underscoring the enhancing effect of li antisense treatment on CTL induction in vivo. This effect may be rather or in addition to enhancing the in vitro expansion of the in vivo activated OVA CTL.

### Example 5

### Enhancement of Protective Antitumor Immunity in Mice Immunized with li AS ODN Treated DC.

To determine whether antisense mediated inhibition of li synthesis in DC can potentiate antitumor immunity, mice (5 mice per group) were immunized twice at weekly intervals with DC transfected with mRNA and treated with ODNs as indicated. 10 days after the second immunization mice were challenged with tumor cells subcutaneously. The results are shown in FIGS. 5A and 5B. Columns represent average tumor volume per group. Referring to FIG. 5A, mice were immunized with DC transfected with either OVA or Flu M1 mRNA, or mock immunized with PBS, treated with either of two li AS ODNs (AE54 or AE40) or two control ODNs (SE46 or SE54), as indicated, and challenged with B16/F101.9-OVA tumor cells. Day 25 tumor measurements are shown. Tumor growth was inhibited in mice immunized with OVA mRNA transfected DC as compared to mice immunized with influenza M1 mRNA transfected DC or injected with PBS. Incubation of the OVA mRNA transfected DC with two li AS ODNs significantly enhanced the antitumor effect whereas two control ODNs had no effect. In this experiment the chicken OVA product was used as a model antigen.

To test whether li inhibition can also potentiate antitumor immunity directed against natural tumor antigens, mice were immunized with TRP-2 mRNA or total F10.9 tumor RNA and challenged with F10.9 tumor. TRP-2 is a melanocyte-specific dominant tumor antigen in the B16 melanoma tumor (Schreurs et al., Cancer Res. 60(24):6995-7001 (2000)). Referring to FIG. 5B, mice were immunized with DC transfected with either TRP2 mRNA, Flu M1 mRNA or total B16/F10.9 tumor RNA (F10.9 RNA) and treated with li AS (AE40) or control (SE40) ODNs, as indicated and challenged with B16/F10.9 tumor cells. Day 23 measurements are shown. As shown in FIG. 5B the antitumor effect seen following vaccination with either TRP-2 or F10.9 mRNA transfected DC was enhanced if the DC were incubated with the li AS ODN but not with a control ODN.

### Example 6

### Enhancement of Tumor Regression in Mice Treated with li AS ODN DC.

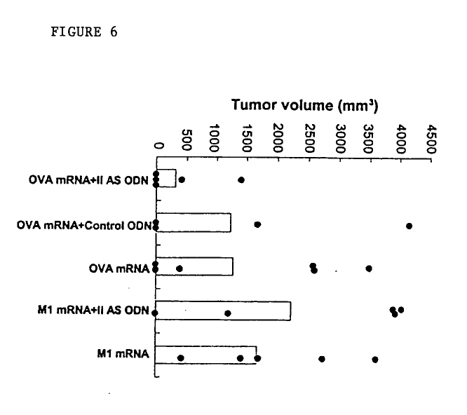

To determine whether li inhibition can also augment protective immunity in the setting of pre-existing tumor, mice (5 mice per group) were implanted with 3×104 B16/F10.9 tumor cells subcutaneously and 2 days later treated with OVA or influenza matrix (M1) mRNA transfected DC exposed to li AS or control ODNs as indicated. Tumor volume was determined 18 days following tumor implantation. FIG. 6 shows that retardation in tumor growth was seen when mice were immunized with OVA mRNA transfected DC, alone or treated with a control ODN, compared to mice immunized with Flu M1 mRNA transfected DC, alone or treated with li As ODN. Retardation of tumor growth was further enhanced when the OVA mRNA transfected DC were treated with the li AS ODNs.

### Example 7

### Experimental Materials and Methods

Mice and Cell Lines

7-8 week old C57BL/6 mice (H-2b) and C3H/He mice (H-2k) were obtained from the Jackson Laboratory, Bar Harbor, Me.

Cell lines used were EL-4 (C57BL/6, H-2b, thymoma), E.G7-OVA (EL4 cells transfected with the OVA cDNA) (Moore et al., Cell 54(6):777-785 (1988)), B16/F10.9 (C57BL/6, H-2b) melanoma tumor cells (Porgador et al., J. Immunogenet. 16(4-5):291-303 (1989), OVA/F10.9, OVA cDNA transfected B16/F10.9 cells, GM-F10.9, B16/F10.9 cells transfected with murine GM-CSF, and RMA-S cells (derived from the Rauscher leukemia virus induced T cell lymphoma RBL-5 of C57BL/6 origin) (Ljunggren and Karre, J. Exp. Med. 162(6):1745-1759 (1985)). The murine MBT-2 cell line derived from a carcinogen-induced bladder tumor in C3H mice (H-2k) (Mickey et al., J. Urol. 127(6):1233-1237 (1982)) was obtained from T. Ratlitt (Washington University, St. Louis, Mo.). Cells were maintained in DMEM supplemented with 10% heat inactivated FCS (GIBCO BRL, Grand Island, N.Y.), 2 mM glutamine, 100 U/ml penicillin and 100 μg/ml streptomycin. E.G7-OVA cells were grown in medium containing 400 μg/ml 6418 (GIBCO BRL, Grand Island, N.Y.). RF33.70 and MF2.2D9 T-cell hybridomas were a kind gift from Dr. Kenneth Rock. These cell lines were maintained in RPMI-1640 supplemented with 10% heat inactivated FCS (GIBCO BRL, Grand Island, N.Y.), 2 mM glutamine, and 100 U/ml penicillin and 100 μg/ml streptomycin.

Peptides and Oligonucleotides (ODNs)

OVA peptide (H-2Kb restricted, SIINFEKL (SEQ ID NO: 1), as 257-264) (Rotzschke et al., Nature 348(6298):252-254 (1990)), VSV peptide (H-2K b restricted, RGYVYGQL; SEQ ID NO: 3) (Burkhart et al., J. Virol. 68(3):1573-1580 (1994)), OVA peptide (I-Ab restricted, IINFEKLTEWTSSNVMEER (SEQ ID NO: 2), as 258-276) (Murphy et al., Science 250(4988):1720-1723 (1990)) and a VSV II peptide (I-Ab-restricted, SSKAQVFEHPHIQDMSQL; SEQ ID NO: 4) (Burkhart et al., J. Virol. 68(3):1573-1580 (1994)) were purchased from Research Genetics (Huntsville, Ala.). Peptides were dissolved in serum-free and protein-free hybridoma media (HYBRIMAX®), Sigma, St Louis, Mo.) and stored at −20° C.

The following phosphorothioate-modified ODNs were synthesized: AE40 (5′-TTG GTG ATC CAT GGC TCT-3′; SEQ ID NO: 5) and AE54 (5′-TGG TCA TCC ATG GCT CTA-3′; SEQ ID NO: 6) correspond to previously described invariant chain (li) antisense ODNs (Qiu et al., Cancer Immunol. Immunother. 48(9):499-506 (1999)). SE40 (5′-TCT CGG TAC CTA GTG GTT-3′ SEQ ID NO: 7), a scrambled sequence of AE40, SE46 (5′-ATG GAT GAC CAA CGC GAC-3′; SEQ ID NO: 8) and SE54 (5′-TAG AGC CAT GGA TGA GGA-3′; SEQ ID NO: 9), the complementary (sense) strand of AE54. All the ODNs were dissolved in OPTI-MEM (GIBCO BRL, Grand Island, N.Y.) at 100 nM and used at 50 nM for electroporation.

Dendritic Cell Generation

DC were generated from bone marrow cultures essentially as previously described (Inaba et al., J. Exp. Med. 176(6):1693-1702 (1992)) with minor modification. Briefly, bone marrow was flushed from the long bones of the limbs and treated with ammonium chloride Tris buffer to deplete red blood cells. Cells were plated at 10⁶ nucleated cells/ml in RPMI-1640 medium supplemented with 5% heat-inactivated FCS, 50 μm 2-mercaptoethanol (2-ME), 10 mm HEPES pH 7.4, Na pyruvate, non-essential amino acids, 2 mm glutamine, 100 units/ml penicillin, 100 μg/ml streptomycin, 10 ng/ml murine IL-4 (PEPERTECH, Rocky Hill, N.J.) and GM-CSF containing medium harvested after 24 hours from GM-F10.9 cells and used at a final dilution of 1:10. At day 3 of culture, floating cells were gently removed and fresh media was added. At day 7 of culture non-adherent cells and loosely adherent aggregates were collected and re-plated in 60 mm Petri dishes (10⁶ cells/ml, 5 ml/dish). Following an additional 2 days of culture, non-adherent cells were removed for analysis. The cells collected at day 7 are enriched for immature DC and the day 9 non-adherent cells consist predominantly of mature DC.

Preparation of Tumor mRNA and In Vitro-transcribed mRNA

Total cellular RNAs from murine melanoma B16/F10.9 and bladder tumor MBT-2 were extracted using the RNeasy kit (Qiagen, Valencia, Calif.) following the manufacturer's specifications. mRNA was isolated from total RNA using MACS mRNA isolation kit (Miltenyi Biotec, Auburn, Calif.) following the manufacturer's instruction. The eluted mRNA was concentrated by ethanol precipitation and dissolving in RNase-free water at 0.5 mg/ml. The plasmids, pGEM4Z/OVA/A64, pGEM4Z/GFP/64A and pGEM4Z/Flu/A6, for in vitro transcription of OVA, GFP and influenza (Flu, M1) mRNA respectively after being linearized by Spe I, were previously described (Nair et al., Nat. Med. 6(9):1011 1017(2000)).

Murine tyrosinase-related protein 2 (TRP2) mRNA was produced by in vitro transcription from a cDNA fragment amplified by RT-PCR using primer pair of: TRP2-T7 (5′-TAATACGACTCACTATAGGGAGACATGGGCCTTGTGGGATGG-3′; SEQ ID NO: 10) and TRP2-64T (5′-T(₆₄)CTAGGCTTCCTCCGTGTATC-3′; SEQ ID NO: 11). First-strand cDNA synthesis was primed with primer TRP2-64T and reverse transcribed using PowerScript reverse transcriptase (BD Bioscience Clontech Palo Alto, Calif.) in 20 μl reaction volume with 0.5 μg B16/F10.9 mRNA for 1 hour at 42° C. TRP2 cDNA was synthesized using Advantage-HF PCR Kit (BD Bioscience Clontech Palo Alto, Calif.) and the following cycling parameters were used: 95° C. for 1 min, 95° C. for 15 s, 58° C. for 30 s, 72° C. for 3 min for 30 cycles. T7 promoter and 64 bp polyA signals were introduced into this TRP2 cDNA, which enable the PCR product being used directed for in vitro transcription without any cloning procedure. The same strategy was also used for a truncated form of OVA (tOVA) of mRNA from which the first 40 as containing leader sequence were removed. The cDNA fragment for tOVA was amplified by RT-PCR using primer pair of T7tOVA (5′-TAATACGACTCACTAGGGAGACATGGCCATGGTATACCTGGGTG-3′; SEQ ID NO: 12) and OVAT64 (5′-T(₆₄AAGGGGAAACACATCTGCC-3′; SEQ ID NO: 13). The sequence of the PCR products were confirmed by DNA sequencing. In vitro transcription was performed using the T7 mMessage mMachine Large Scale In Vitro Transcription kit (Ambion, Austin, Tex.). Briefly, transcription mix, ribonucleotide mix, purified cDNA, and T7 enzyme mix were mixed and incubated at 37° C. for 2 hours. The DNA template was degraded by adding DNase I (RNase free) and incubating at 37° C. for 15 min. The RNA was purified using RNeasy Columns (Qiagen) according the manufacturer's protocol. All the mRNAs were eluted in RNase-free water at 0.5 g/ml and stored at −80° C. for further use.

Electroporation of DC

Electroporation of murine DC was adapted from the protocol described for human DC (Van Tendeloo et al., Blood 98(1):49-56 (2001)). Briefly, prior to electroporation, day 9 DC were washed twice with serum-free Opti-MEM and resuspended to a final concentration of 2.5×10⁷ cells/mL in Opti-MEM. Subsequently, 0.05 to 0.2 mL of the cell suspension was mixed with mRNA (2 μg per 1×10⁶ cells) and ODN at 50 nM (when indicated), and electroporated in a 0.2-cm cuvette using an ECM803 Electroporator (BTX, a division of Genetronics, Inc. San Diego Calif.). After electroporation, conditioned medium prepared by mixing DC culture medium with supernatant collected from day 7 DC (1:1) was added to the cell suspension and cells were further incubated at 37° C. in a humidified atmosphere supplemented with 5% CO₂ for at least 1 hour.

Flow Cytometry

All monoclonal antibodies (mAbs) were purchased from PharMingen (San Diego, Calif.). Anti-CD40, CD80, CD86, I-Ab, and CD74 (ii) were conjugated with FITC and anti-CD11c mAB was conjugated with PE. Cells were stained by incubation with mAb for 30 min at 4° C. in FRCS buffer (PBS/3% FCS/0.01% azide), washed with PBS twice, and analyzed on a FACScalibur flow cytometer (Becton Dickinson, San Jose, Calif.). Intracellular staining of CD74 was performed using Cytofix/Cytoperm Plus (with ColgiStop) Kit (PharMingen, San Diego, Calif.), following manufacturers' instruction.

Antigen Presentation In Vitro

Day 9 DC were electroporated with OVA mRNA in the presence of ODNs where indicated. After 1-2 hours incubation at 37° C., DC were washed with PBS and incubated for 20-24 hours with 1×10⁵ RF33.70 T-cell hybridomas specific to the H-2Kb⁺ OVA 257-264 peptide or MF2.2D9 T-cell hybridoma specific for I-Ab⁺ OVA 258-276 peptide at indicated DC/hybridoma ratio. The T-hybridomas were a gift of Dr. Kenneth Rock. T-cell stimulation was measured by testing supernatants for interleukin-2 (IL-2) content by enzyme-linked immunosorbent assay (ELISA) according to the manufacturer's instructions (Endogen, Cambridge, Mass.).

OVA CD4⁺ T Cell Proliferation Assay

Mice were immunized i.v. with 2-4×10⁵ electroporated DCs in 200 μl of PBS. Splenocytes were harvested after 8 days and CD4⁺ T cells were isolated using StemSep Murine CD4 Negative Isolation Column (StemCell Technologies Inc.). CD4⁺ T cells were co-cultured with I-Ab-restricted OVA 258-276 peptide or VSV peptide pulsed DC for 3 days. ³H-thymidine incorporation was measured for 17 hours prior to harvest.

CTL Induction In Vivo and Cytotoxicity Assay

Mice were immunized i.v. with 2.5×10⁵ electroporated DC in 200 μl of PBS. Splenocytes were harvested after 8 days and depleted of red blood cells (RBCs) with ammonium chloride/Tris buffer. Splenocytes (1×10⁷) were cultured with 2×10⁵ OVA mRNA electroporated DC in 5 ml of IMDM with 10% FCS, 1 mM sodium pyruvate, 100 lU/ml penicillin, 100 mg/ml streptomycin, and 5×10⁻⁵ M 2-ME per well in a 6-well tissue culture plate. Cells were cultured for 5 days at 37° C. and 5% CO₂. Effectors were harvested on day 5 on Histopaque 1083 gradient before use in a CTL assay.

4×10⁶ target cells were labeled with ⁵¹Cr for 60-90 minutes at 37° C. After several washes, 10⁴ Cr-labeled targets and serial dilutions of effector cells at varying effector: target ratios were incubated in 200 μl of RPMI-1640 with 10% heat-inactivated FCS in 96 well V-bottom plates. The plates were centrifuged at 500 g for 3 minutes and incubated at 37° and 5% CO₂ for 4 hours. 100 μl of the supernatant was harvested and chromium release was measured in a scintillation counter. Specific cytotoxic activity was determined using the following formula: % specific release={(experimental release—spontaneous release)/(total release—spontaneous release)}×100. Spontaneous release of the target cells was <10% of total release by detergent in all assays. SE of the means of triplicate cultures was <5%

In Vivo Depletion of CD4 or CD8 T Cells

Mice were injected i.p. with 150 μg anti-CD4 ascites (GK1.5) or 200 μg anti-CD8 (53-6.73) at days −3, 0, +3, and +6 of tumor challenge. Greater than 98% and 94% of CD4⁺ or CD8⁺ T cells were specifically depleted under those conditions, respectively. Non-depleted control mice received injection of 200 μg mouse IgG.

Tumor Challenge

Mice were immunized twice i.p. with 2-4×10⁵ electroporated DC in 200 μl PBS weekly. After 10 days, the mice were challenged subcutaneously with 1×10⁵ B16/F10.9-OVA (for OVA immunized the mice) or, B16/F10.9 (for TRP2 or tumor mRNA immunized the mice) or, 3×10⁵ MBT-2 (for MBT-2 mRNA immunized mice) in 200 μl PBS. For the treatment of pre-existing tumor, 3×10⁴ F10.9-OVA was subcutaneously injected at the right flank of C57BL/6 mice. The mice were than immunized i.p. with 5×10⁵ RNA electroporated DC at 3, 7, 14, and 21 days respectively after tumor inoculation. Tumor growth was measured using a caliper every other day starting at day 13 after tumor challenge. Mice were sacrificed when the diameter of the tumor reached 3 cm.

All documents cited above are hereby incorporated in their entirety by reference.

It will be understood that various details of the invention may be changed without departing from the scope of the invention. Furthermore, the foregoing description is for the purpose of illustration only, and not for the purpose of limitation.

What is claimed is:

1. An immunogenic composition comprising antigen presenting cells containing a nucleic acid encoding at least one antigen, said antigen presenting cells further comprising an inhibitor of invariant chain (li).

2. The immunogenic composition of claim 1 wherein the inhibitor of invariant chain is an antisense oligonucleotide.

3. The immunogenic composition of claim 1 wherein the inhibitor of invariant chain is an RNAi molecule.

4. The immunogenic composition of claim 1 wherein the inhibitor is a ribozyme that specifically cleaves the li mRNA.

5. The immunogenic composition of claim 1 wherein the inhibitor is a molecule which inhibits the interaction of li protein with MHC class II molecules.

6. The immunogenic composition of claim 1 wherein the nucleic acid is mRNA encoding at least one tumor antigen.

7. The immunogenic composition of claim 1, wherein the nucleic acid is mRNA encoding a pathogen antigen.

8. The immunogenic composition of claim 6 wherein the nucleic acid comprises total tumor RNA.

9. An antigen presenting cell (APC) transfected with mRNA encoding at least one antigen, said APC expressing on its surface said antigen in association with an MHC Class II molecule.

10. The antigen presenting cell of claim 9, wherein invariant chain expression or function is inhibited.

11. A method of enhancing MHC class II presentation of epitopes encoded by mRNA transfected into antigen presenting cells comprising treating the antigen presenting cells with an inhibitor of invariant chain.

12. The method according to claim 11, wherein the inhibitor downregulates expression of invariant chain.

13. The method according to claim 11, wherein the inhibitor inhibits the function of invariant chain.

14. A method for enhancing a CD4 T cell response to an antigen comprising transfecting antigen presenting cells with mRNA encoding said antigen and treating said cells with an inhibitor of li expression.

15. A method for the treatment of cancer comprising administering to a patient in need, a therapeutic dose of antigen presenting cells transfected with mRNA encoding at least one tumor antigen wherein expression of invariant chain in said antigen presenting cells has been depressed.

16. A method of modulating an immune response to an antigen comprising:
(a) transfecting antigen presenting cells (APC) with mRNA encoding the antigen and downregulating the expression of invariant chain to provide modified APC, and
(b) administering said APC to a patient in need.

17. A method according to claim 17 wherein the antigen is a tumor antigen, a pathogen antigen or an antigen associated with an autoimmune disease.

18. A kit for the preparation of modified antigen presenting cells (APC) comprising:
(a) tumor RNA, and
(b) an li inhibitor.
